SEQUENCE LISTING (6 sequences)

SEQ ID NO: 1 - protein, 559 aa, Protaminobacter rubrum
MTIPKWWKEAVFYQVYPRSFKDTNGDGIGDINGIIEKLDYLKALGIDAIWINPHYDSPNTDNGYDIRDYRKIMKEYGTMEDFDRLISEMKKRNMRLMIDVVINHTSDQNEWFVKSKSSKDNPYRGYYFWKDAKEGQAPNNYPSFFGGSAWQKDEKTNQYYLHYFAKQQPDLNWDNPKVRQDLYAMLRFWLDKGVSGLRFDTVATYSKIPDFPNLTQQQLKNFAAEYTKGPNIHRYVNEMNKEVLSHYDIATAGEIFGVPLDQSIKFFDRRRDELNIAFTFDLIRLDRDSDQRWRRKDWKLSQFRQIIDNVDRTAGEYGWNAFFLDNHDNPRAVSHFGDDRPQWREPSAKALATLTLTQRATPFIYQGSELGMTNYPFKAIDEFDDIEVKGFWHDYVETGKVKADEFLQNVRLTSRDNSRTPFQWDGSKNAGFTSGKPWFKVNPNYQEINAVSQVTQPDSVFNYYRQLIKIRHDIPALTYGTYTDLDPANDSVYAYTRSLGAEKYLVVVNFKEQMMRYKLPDNLSIEKVIIDSNSKNVVKKNDSLLELKPWQSGVYKLNQ

SEQ ID NO: 2 - protein, 578 aa, Pantoea dispersa
MASPLTKPSTPIAATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGKDNPYRDYYFWRDGKQGQAPNNYPSFFGGSAWQLDKQTDQYYLHYFAPQQPDLNWDNPKVRAELYDILRFWLDKGVSGLRFDTVATFSKIPGFPDLSKAQLKNFAEAYTEGPNIHKYIHEMNRQVLSKYNVATAGEIFGVPVSAMPDYFDRRREELNIAFTFDLIRLDRYPDQRWRRKPWTLSQFRQVISQTDRAAGEFGWNAFFLDNHDNPRQVSHFGDDSPQWRERSAKALATLLLTQRATPFIFQGAELGMTNYPFKNIEEFDDIEVKGFWNDYVASGKVNAAEFLQEVRMTSRDNSRTPMQWNDSVNAGFTQGKPWFHLNPNYKQINAAREVNKPDSVFSYYRQLINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 3 - protein, 571 aa, Raoultella planticola
MAPSVNQNIHVHKESEYPAWWKEAVFYQIYPRSFKDTNDDGIGDIRGIIEKLDYLKSLGIDAIWINPHYDSPNTDNGYDISNYRQIMKEYGTMEDFDNLVAEMKKRNMRLMIDVVINHTSDQHPWFIQSKSDKNNPYRDYYFWRDGKDNQPPNNYPSFFGGSAWQKDAKSGQYYLHYFARQQPDLNWDNPKVREDLYAMLRFWLDKGVSSMRFDTVATYSKIPGFPNLTPEQQKNFAEQYTMGPNIHRYIQEMNRKVLSRYDVATAGEIFGVPLDRSSQFFDPRRHELNMAFMFDLIRLDRDSNERWRHKSWSLSQFRQIISKMDVTVGKYGWNTFFLDNHDNPRAVSHFGDDRPQWREASAKALATITLTQRATPFIYQGSELGMTNYPFRQLNEFDDIEVKGFWQDYVQSGKVTATEFLDNVRLTSRDNSRTPFQWNDTLNAGFTRGKPWFHINPNYVEINAEREETREDSVLNYYKKMIQLRHHIPALVYGAYQDLNPQDNTVYAYTRTLGNERYLVVVNFKEYPVRYTLPANDAIEEVVIDTQQQATAPHSTSLSLSPWQAGVYKLR

SEQ ID NO: 4 - protein, 562 aa, Pseudomonas mesoacidophila
MEEAVKPGAPWWKSAVFYQVYPRSFKDTNGDGIGDFKGLTEKLDYLKGLGIDAIWINPHYASPNTDNGYDISDYREVMKEYGTMEDFDRLMAELKKRGMRLMVDVVINHSSDQHEWFKSSRASKDNPYRDYYFWRDGKDGHEPNNYPSFFGGSAWEKDPVTGQYYLHYFGRQQPDLNWDTPKLREELYAMLRFWLDKGVSGMRFDTVATYSKTPGFPDLTPEQMKNFAEAYTQGPNLHRYLQEMHEKVFDHYDAVTAGEIFGAPLNQVPLFIDSRRKELDMAFTFDLIRYDRALDRWHTIPRTLADFRQTIDKVDAIAGEYGWNTFFLGNHDNPRAVSHFGDDRPQWREASAKALATVTLTQRGTPFIFQGDELGMTNYPFKTLQDFDDIEVKGFFQDYVETGKATAEELLTNVALTSRDNARTPFQWDDSANAGFTTGKPWLKVNPNYTEINAAREIGDPKSVYSFYRNLISIRHETPALSTGSYRDIDPSNADVYAYTRSQDGETYLVVVNFKAEPRSFTLPDGMHIAETLIESSSPAAPAAGAASLELQPWQSGIYKVK

SEQ ID NO: 5 - protein, 576 aa, Enterobacter sp.
MAYSAETSVTQSIQTQKESTLPAWWKEAVFYQIYPRSFKDTNGDGIGDIRGIIEKLDYLKSLGIDAIWINPHYDSPNTDNGYDIRDYEKIMQEYGTMEDFDTLVSEMKKRNMRLMIDVVINHTSDQHPWFIQSKSSKENPYREYYFWRDGKDNQPPNNYPSFFGGSAWQKDDKTGQYYLHYFARQQPDLNWDNPKVRGDLYAMLRFWLDKGVSGMRFDTVATYSKIPGFPDLTPEQQKNFAEQYTTGPNIHRYLQEMKQEVLSRYDVVTAGEIFGVPLERSSDFFDRRRNELDMSFMFDLIRLDRDSNERWRHKKWTLSQFRQIINKMDSNAGEYGWNTFFLDNHDNPRAVSHFGDDSPQWIEPSAKALATIILTQRATPFIFQGSELGMTNYPFKKLNEFDDIEVKGFWQDYVQTGKVSAEEFIDNVRLTSRDNSRTPFQWNDRKNAGFTSGKPWFRINPNYVEINADKELIRNDSVLNYYKEMIKLRHKTPALIYGTYKDISPEDDSVYAYTRTLGKERYLVVINFTEKTVRYPLPENNVIKSILIEANQNKTAEKQSTVLTLSPWQAGVYELQ

SEQ ID NO: 6 - protein, 578 aa, Pectobacterium carotovorum
MATNHNEQDTKTVIAVNDGVSAHPVWWKEAVFYQVYPRSFKDSNGDGIGDLKGLTEKLDYLKTLGINAIWINPHYDSPNTDNGYDIRDYRKIMKEYGTMDDFDNLIAEMKKRDMRLMIDVVVNHTSNEHKWFVESKKSKDNPYRDYYIWRDGKDGTPPNNYPSFFGGSAWQKDNVTQQYYLHYFGVQQPDLNWDNPKVREEVYDMLRFWIDKGVSGLRMDTVATFSKNPAFPDLTPEQLKNFAYTYTQGPNLHRYIQEMHQKVLAKYDVVSAGEIFGVPLEEAAPFIDQRRKELDMAFSFDLIRLDRAVEERWRRNDWTLSQFRQINNRLVDMAGQYGWNTFFLSNHDNPRAVSHFGDDRPEWRIRSAKALATLALTQRATPFIYQGDELGMTNYPFTSLSEFDDIEVKGFWQDFVETGKVKPDVFLENVKQTSRDNSRTPFQWSNAEQAGFTTGTPWFRINPNYKNINAEDQTQNPDSIFHFYRQLIALRHATPAFTYGAYQDLDPNNNEVLAYTRELNQQRYLVVVNFKEKPVHYALPKTLSIKQTLLESGQKDKVAPNATSLELQPWQSGIYQLN

(19) United States
(12) Patent Application Publication
(10) Pub. No.: US 2025/0177500 A1
(43) Pub. Date: Jun. 5, 2025

(54) SUCROSE ISOMERASES AS FOOD AND NUTRITIONAL SUPPLEMENTS

(71) Applicant: DSM IP ASSETS B.V., Heerlen (NL)

(72) Inventors: Maaike Johanna BRUINS, Kaiseraugst (CH); Petrus Jacobus Theodorus DEKKER, Kaiseraugst (CH); Jeroen Adrianus Johannes NOOIJENS, Kaiseraugst (CH)

(21) Appl. No.: 19/038,830

(22) Filed: Jan. 28, 2025

Related U.S. Application Data
(62) Division of application No. 17/269,951, filed on Feb. 19, 2021.

(30) Foreign Application Priority Data
Aug. 22, 2018 (EP) 18190215.6

(51) Int. Cl. A61K 38/52; A23K 20/189; A23L 2/52; A23L 29/00; A23L 33/00; A23L 33/10

(52) U.S. Cl. CPC A61K 38/52; A23K 20/189; A23L 2/52; A23L 29/06; A23L 33/10; A23L 33/40; A23V 2002/00

(57) ABSTRACT

Sucrose isomerase is used as a nutritional supplement, or can be mixed in with a powderous food/beverage formulation. When an animal, including a human, consumes sucrose, the sucrose isomerase will act on the sucrose present in the food, and will convert the sucrose to other sugars. This results in lowering of the glycemic index of the food without changing the formulation of the food.

### CROSS-REFERENCE TO RELATED APPLICATIONS

This application is a divisional of U.S. patent application Ser. No. 17,269,951, filed on Feb. 19, 2021, which is the U.S. national phase of International Application No. PCT/EP2019/072310 filed Aug. 21, 2019 which designated the U.S. and claims priority to EP 18190215.6 filed Aug. 22, 2018, the entire contents of each of which are hereby incorporated by reference.

### REFERENCE TO SEQUENCE LISTING SUBMITTED ELECTRONICALLY

The content of the electronically submitted sequence listing (Name 4662_4784_Sequence Listing.xml; Size: 9,607 bytes and Date of Creation: Jan. 27, 2025) is incorporated by reference in its entirety.

### FIELD OF THE INVENTION

This invention relates to the use of sucrose isomerases as a nutritional supplement for both humans and animals. The sucrose isomerases can enzymatically reduce the amount of sucrose in foodstuff after it is consumed, and thus lower the glycemic index of foods. The sucrose isomerase supplements are of particular benefit for lowering blood sugars, lowering the glycemic index of food and/or beverages consumed, and managing or losing weight.

### BACKGROUND TO THE INVENTION

High blood glucose ranks very high in the global burden of disease cause and can lead to obesity and diabetes type 2. Foods and beverages containing rapidly absorbed carbohydrates like sugar or starch can lead to a fast increase in blood glucose, following by a spike in the insulin release leading to a fast decrease in blood glucose. Foods and beverages lacking such carbohydrates give a slower, and lower, increase in blood glucose, without the rapid spike in insulin release. This response in blood glucose is expressed as the Glycemic Index (GI) of a food, that is expressed relative to the response towards the intake of a reference containing glucose or white bread (set at 100). For many foods the GI has been determined. Diets based on carbohydrate foods that are more slowly digested, absorbed, and metabolized (i.e., low-GI diets) have been shown to give better insulin sensitivity than high-GI diets. Low GI-diets are associated with reduced risk of type-2 diabetes and cardiovascular disease as compared to high-GI diets. A role of low-GI compared to high-GI diets in satiety, weight maintenance and prevention of diet-related diseases has been suggested. Glycemic load (GL) is calculated by multiplying the grams of available carbohydrate in the food times the food's GI and then dividing by 100.

Sucrose is abundant in the diet and constitutes ˜35-40% of all carbohydrates in the diet. A challenge to those that wish to lower the glycemic load, is that many preferred indulgent foods and beverages contain high amounts of sucrose. For example, some candy bars contain up to 30 grams sucrose (50% on weight); a can (330 cc) of cola contains 39 grams sucrose; chocolate milk has 58 grams sucrose in 450 cc; and ice cream often contains 28% sucrose on weight. However, reducing the sugar content in these foods often negatively impacts the sweetness, mouth-feel and indulgent character. Replacing the sucrose in such products by other sugars with lower GI (e.g. tagatose, allulose or palatinose), sugar alcohols (e.g. sorbitol, mannitol, or xylitol), or by high potency sweeteners (e.g. aspartame, sucralose or stevia) will either lead to a less sweet final product, or have a negative effect on textural properties and/or taste of the food or beverage, and thereby reduce the indulgent character of the product. Therefore, such sucrose replacements are not used frequently in food products, and their use is mainly focused on sweetened beverages.

Isomaltulose is available to the food industry from Beneo under the tradename Palatinose™ and used in foods as sugar replacement. Palatinose™ is fully available in the small intestine, but is hydrolyzed 4-5 times more slowly, leading to a low glycemic response and to lower insulin levels. It has been shown that the substitution of sucrose by Palatinose™ leads to lower insulin peaks and increased fat burning on exercise. Beneo markets Palatinose™ for weight management, its non-cariogenic character, enhanced endurance performance in sports, prevention of gestational diabetes, sustained cognitive functioning, and improvement of the metabolic profile in elderly (Beneo-institute, 2017 beneo.com/Expertise/BENEO-Institute/News_Papers/BENEO_paper_palatinose_US_201708v1_web_USLetter_1.pdf).

For trehalulose (in patent literature also called “Vitalose”) similar benefits are envisaged, since it is also slowly digested by the intestinal sucrase, but less clinical data is available. Blood glucose levels and insulin response is similar or even slightly more moderate than that of isomaltulose (as reported in European Patent EP2418971B1).

An inhibitory effect of these sugars on the activity of the sucrase/amylase activity in the small intestine is suggested in literature (Kashimura & al, 2008 J. Agric. Food Chem. 56:5892-5898), but solid proof for this is still missing. Such inhibitory effect may slow the conversion of sucrose and starch and thereby further reduce the glycemic load. Hence, isomaltulose and/or trehalulose consumption may have an additional benefit on reduction of the glycemic load, by inhibiting sucrase/amylase activity in the intestine, when combined with starch-containing foods.

One problem with the use of either isomaltulose or trehalulose in foods and beverages is that the sweetness of these sugars is much lower than that of sucrose on a per gram basis. Replacement of sucrose by these sucrose isomers would therefore require drastic changes in the composition of the food or beverage. The lower sweetness has to be compensated for by e.g. addition of extra artificial sweeteners, which may affect the taste of the final product. Also, isomaltulose is relatively expensive compared to sucrose and therefore there may be economic reasons not to add it into the food or beverage product.

Therefore, there is a need in society for sweet, indulgent foods and beverages that will not lead to a high glycemic response after consumption. Conscious consumers may want to prevent high glycemic index and blood sugar increase after consuming sucrose-containing foods/drinks. Moreover, conversion of high glycemic index carbohydrates into low glycemic index carbohydrates in the stomach has a clinically significant benefit for glycemic control in people with type 1 and type 2 diabetes. Glycemic index lowering nutrition therapy can reduce glycated hemoglobin (A1C) in type 2 diabetes persons by 1.0% to 2.0% and, when used with other components of diabetes care, can further improve clinical and metabolic outcomes.

### DETAILED DESCRIPTION OF THE INVENTION

We surprisingly found that using the enzyme sucrose isomerase as a nutritional supplement or as part of a medical diet or medical diet supplement will lower the sucrose content of sweet foods and/or beverages when consumed together with the enzyme. Sucrose isomerase used as nutritional supplement or as part of a dry food or beverage (such as a premix or the like) will therefore lower the glycemic index of such foods in the intestinal tract, without affecting the composition and properties of the food or beverage before consumption. Consequently, sucrose isomerase as a nutritional supplement may be used to reduce the risk of type-2 diabetes and cardiovascular disease without changing eating habits. Also, by lowering the glycemic load, sucrose isomerase as nutritional supplement may fit in programs for weight maintenance and may prevent high-sugar diet-related diseases. Sucrose isomerase as nutritional supplement may also be useful for sports nutrition by slowing down the uptake of sugars. Sucrose isomerase can also be included in a ready-to-mix meal replacer for diabetics or prediabetics who are advised or prescribed a low carbohydrate diet, without disturbing the carbohydrate content of the meal replacer.

Thus, one embodiment of this invention is a method of reducing insulin levels in an animal, including a human, who consumes a food or drink comprising sucrose, the method comprising administering to the animal or human an effective amount of a sucrose isomerase nutraceutical, dietary supplement, or pharmaceutical prior to, or commensurate with the consumption of the food or drink. Another embodiment of this invention is a method of lowering the glycemic index of a food or drink comprising sucrose consumed by an animal, including a human, comprising administering to the animal or human sucrose isomerase in the form of a nutraceutical, dietary supplement, or pharmaceutical. Another embodiment of this invention is the use of sucrose isomerase to manufacture a nutritional supplement, dry and/or powdered food or drink, or pharmaceutical which lowers the glycemic index of a food or drink. Another embodiment of this invention is sucrose isomerase as a nutritional supplement which lowers the glycemic index of a food or drink.

New research suggests that the key to sustained endurance for athletes might not lie in the consumption of the so-called “quick carbohydrates”, but in “slower carbohydrates” that balance blood sugars instead of providing a rush of energy in the form of blood glucose. For purposes of this invention, “endurance exercise” means that the exercise is one which is increases breathing and heart rate, such as walking, jogging, swimming, or biking or the like. Thus, another embodiment of this invention is a method of slowing or sustaining sugar absorption over a period of time to enhance an athlete's ability to perform an endurance exercise comprising administering an effective amount of a sucrose isomerase to a person performing the endurance exercise who also consumes food or drink containing sucrose during the endurance exercise. Yet another embodiment of this invention is a method of enhancing sustained endurance in an athlete comprising administering an effective amount of a sucrose isomerase to a person engaged in an athletic endurance activity who also consumes food or drink containing sucrose. Another embodiment of this invention is the use of sucrose isomerase to increase a person's ability to perform an endurance exercise.

Another embodiment of this invention is a method of sustaining and/or slowing sugar absorption to sustain energy release and to minimize the blood glucose rise and the so-called after-meal “dip” after a sucrose-containing meal comprising administering an effective amount of a sucrose isomerase to a healthy or (pre) diabetic person who also consumes food containing sucrose. This is particularly beneficial in a situation where a person consumes a meal (such as a mid-day meal) and wants to remain alert and avoid a period of drowsiness a few hours after consumption.

Another embodiment of this invention is a method of assisting an animal, including a human to lose weight or maintain a weight loss comprising administering to the animal or person an effective amount of a sucrose isomerase.

In one embodiment, sucrose isomerase is used in human nutrition.

In another embodiment, the sucrose isomerase is used to benefit an animal, preferably a companion animal (such as cats, dogs, equines, and domesticated pigs typically used as pets) who may consume sucrose. Companion animals are often prone to obesity and suffer from its adverse consequences. The sucrose isomerases of this invention offer a way to combat diabetes, weight gain, and associated metabolic disorders in companion animals without resorting to expensive and inconvenient insulin injections.

It is preferred that the sucrose isomerase is taken at least once a day prior to consumption of the food or drink containing sucrose. It is also preferred that it is taken shortly before (i.e. less than one hour prior to consumption, and more preferably less than 30 minutes prior to consumption. It is particularly preferred that it is taken immediately prior or during the consumption). In another embodiment, the sucrose isomerase is taken within 2 hours of eating a meal.

### DESCRIPTION OF THE FIGURES

FIG. 1 is a readout of an HPLC used to separate sugars as detailed in Example 1.

Sucrose isomerase is used in the industrial production of isomaltulose from sucrose. To our knowledge, there has been no description of use of sucrose isomerase as a supplement, where the sucrose isomerase can survive both the processes involved in creating a tablet or other suitable formulation as well as the human digestive process, so that it still remains active in the stomach and/or digestive tract. While sucrose isomerases are known in the art, their activity has only been observed in the context of laboratory buffers.

A sucrose isomerase converts sucrose (2-O-α-D-Glucopyranosyl-D-fructose) into the lower glycemic sugars isomaltulose (6-O-α-D-Glucopyranosyl-D-fructose) and/or trehalulose (1-O-α-D-Glucopyranosyl-D-fructose) (Mu & al (2014) Appl Microbiol Biotechno/98:6569-6582).

As shown in EXAMPLES 4-6, another enzyme, glycosyl transferase, which also acts on sucrose, albeit via a different mechanism, was found to be inactive when subjected to conditions mimicking the human digestive system. Thus, it is not predictable that enzymes which use sucrose as a substrate will be suitable for use as nutritional supplements.

The sucrose isomerase of this invention may be from any source, provided that it is robust enough to survive the formulation and digestive process well enough so that an effective amount is available to act on ingested sugars. There are at least 5 art-recognized classes of the sucrose isomerase (see Goulter et al 2012 Enz and Microb Technol 50:57-64):
Group I: includes Serratia plymuthica, and Protaminobacter rubrum
Group II which includes Erwinia rhapontici
Group III which includes Enterobacter sp, Roaultella planticola, and Klebsiella singaporensis
Group IV which includes Pantoea dispersa and
Group V which includes Pseudomonas mesoacidophilia and Rhizobium sp.

Examples of preferred sucrose isomerases include those found in:
Protaminobacter rubrum, including the enzyme identified as Uniprot: DOVX20,
Pantoea dispersa, including the enzyme identified as Uniprot: Q6XNK6,
Raoultella planticola, including the enzyme identified as Uniprot: Q6XKX6,
Pseudomonas mesoacidophila, including the enzyme identified as Uniprot: Q2PS28,
Enterobacter including the enzyme identified as Uniprot: B5ABD8; and
Pectobacterium carotovorum including the enzyme identified as Uniprot: S5YEW8.

When present, their signal peptides were replaced with a Methionine (M) and this resulted in the protein sequences depicted in SEQ ID NO:1-6, respectively.

Preferred sucrose isomerases include those identified in the Examples as Sis4, Sis10, Sis 12, Sis14 and Sis15. A particularly preferred sucrose isomerase is Sis4.

The invention as described here circumvents the prior problems of the use of isomaltulose, trehalulose or any other low-glycemic sugar replacer, in food and beverage formulations. By supplying sucrose isomerase as nutritional ingredient, the food or beverage formulation does not need to be changed and isomaltulose and/or trehalulose are only formed during digestion in the stomach or the upper intestinal tract.

Preferred sucrose-containing foods/beverages relevant for this invention include indulgent foods such as:
Desserts—ice-cream, pudding, custard, yoghurt
Confectionary—sweets, chocolate
Baking—cookies, pastry, pies, donuts, breakfast cereals
Beverages—soft drinks, energy drinks, fruit juices, flavored milk
Fruits and vegetables—corn, tropical fruits, dates, banana, beetroot, pumpkin
Spreads—jams, marmalade, chocolate spread, peanut butter.
Food for companion animals: in the form of treats or chewy snacks

### Formulations

The dietary and pharmaceutical compositions according to the present invention may be in any galenic form that is suitable for administering to the body especially in any form that is conventional for oral administration, e.g. in solid form, such as additives/supplements for food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be encapsulated in hard or soft shell capsules, whereby the capsules feature e.g. a matrix of fish, swine, poultry, or cow gelatin, plant proteins or ligninsulfonate. The dietary and pharmaceutical compositions may be in the form of controlled or delayed release formulations. The compositions of the present invention are not administered topically, such as application to the nasal passage.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

In addition, compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

### Dosages

Dosage of the enzyme as a nutritional supplement are 0.1-500 mg of pure sucrose isomerase protein per 100 g ingested sucrose, preferably 0.5-100 mg, 2-50 mg, 10-25 mg sucrose isomerase per 100 g ingested sucrose. The dosage will, of course vary depending on how much sucrose is ingested per day or per meal or per beverage. For example, if a person consumes 75 grams of added sucrose per day, which is a common amount in some western countries, then a preferred amount of daily enzyme would be 7.5-20 mg pure sucrose isomerase protein. For example, if a person drinks a 300 ml beverage containing 100 g/l sucrose, a preferred amount of enzyme would be 3-7.5 mg pure sucrose isomerase protein, to be taken together with the beverage.

A typical composition with sucrose isomerase may contain silicon dioxide, (Micro) Cellulose (e.g. Avicel PH102), magnesium stearate, stearic acid, polyvinyl pyrrolidone (e.g. Crospovidone) and/or maltodextrin. Per tablet of 300 mg such composition may contain e.g. 60 mg of a dried enzyme formulation (e.g. containing 7.5-20 mg pure sucrose isomerase plus maltodextrin until 60 mg), 15 mg crospovidone, 2.5 mg magnesium stearate and 222.5 mg Avicel PH102.

In addition, the composition may be a dry food, soft-drink powder or meal replacement powder. Such dry composition typically has a water activity (Aw) of <0.5. A typical isotonic sports energy drink powder may contain up to 90 g carbohydrates per 100 g powder, of which 75 g may be sugar (mainly sucrose and glucose). Additionally, the powder will contain 2.15 g mineral salts per 100 g (mainly potassium chloride, potassium citrate, sodium citrate, sodium chloride, and magnesium citrate), besides some citric acid, flavourings and colour. Preferably sucrose isomerase is added at 7.5-20 mg pure dry enzyme per 100 g of such powder.

### Enzymes

### Homology

For the purpose of this disclosure, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this disclosure the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the disclosure is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The nucleic acid and protein sequences of the present disclosure can further be used as a “query sequence” to perform a search against public databases to, for example, identify other family members or related sequences. Such searches can be performed using the BLASTN and BLASTX programs (version 2.0) of Altschul, et al. (1990) J. Mol. Biol. 215:403-10. BLAST nucleotide searches can be performed with the BLASTN program, score=100, wordlength=12 to obtain nucleotide sequences homologous to nucleic acid molecules of the disclosure. BLAST protein searches can be performed with the BLASTX program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTX and BLASTN) can be used. See the homepage of the National Center for Biotechnology Information at ncbi.nlm.nih.gov/.

As used herein, the terms “variant, “derivative”, “mutant” or “homologue” can be used interchangeably. They can refer to either polypeptides or nucleic acids. Variants include substitutions, insertions, deletions, truncations, transversions, and/or inversions, at one or more locations relative to a reference sequence. Variants can be made for example by site-saturation mutagenesis, scanning mutagenesis, insertional mutagenesis, random mutagenesis, site-directed mutagenesis, and directed-evolution, as well as various other recombination approaches. Variant polypeptides may differ from a reference polypeptide by a small number of amino acid residues and may be defined by their level of primary amino acid sequence homology/identity with a reference polypeptide. Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a reference polypeptide. Methods for determining percent identity are known in the art and described herein. Generally, the variants retain the characteristic nature of the reference polypeptide, but have altered properties in some specific aspects. For example, a variant may have a modified pH optimum, a modified substrate binding ability, a modified resistance to enzymatic degradation or other degradation, an increased or decreased activity, a modified temperature or oxidative stability, but retains its characteristic functionality. Variants further include polypeptides with chemical modifications that change the characteristics of a reference polypeptide.

With regard to nucleic acids, the terms refer to a nucleic acid that encodes a variant polypeptide, that has a specified degree of homology/identity with a reference nucleic acid, or that hybridizes under stringent conditions to a reference nucleic acid or the complement thereof. Preferably, a variant nucleic acid has at least 70%, at least 75%, at least 80%, at least 85%, at least 90%, at least 91%, at least 92%, at least 93%, at least 94%, at least 95%, at least 96%, at least 97%, at least 98%, or even at least 99% nucleic acid sequence identity with a reference nucleic acid. Methods for determining percent identity are known in the art and described herein.

The present invention is further illustrated by the following Examples

### EXAMPLES

### Example 1

### Sucrose Isomerases

Six proteins annotated as sucrose isomerases were selected from the Uniprot data base. The sequences originated from Protaminobacter rubrum (Uniprot: DOVX20), Pantoea dispersa (Uniprot: Q6XNK6), Raoultella planticola (Uniprot: Q6XKX6), Pseudomonas mesoacidophila (Uniprot: Q2PS28), Enterobacter (Uniprot: B5ABD8), and Pectobacterium carotovorum (Uniprot: S5YEW8). Putative signal peptides were predicted by SignalP 4.1 prediction software for gram negatives (Petersen, Nature Methods, 8:785-786, 2011). When present these signal peptides were replaced with a Methionine (M) and this resulted in the protein sequences depicted in SEQ ID NO:1-6.

The protein sequences (SEQ ID NO:1-6) were expressed in E. coli as described in WO2017050652 (A1). Synthetic DNA sequences encoding the putative sucrose isomerases were codon optimized for expression in E. coli according to the algorithm of DNA2.0 (GeneGPS® technology). For cloning purposes, DNA sequences containing a NdeI site was introduced at the 5′-end and a DNA sequence containing a stop codon and an AscI site was introduced at the 3′end. The synthetic DNA encoding the putative sucrose isomerases were cloned via the 5′NdeI and 3′ AscI restriction sites into an arabinose inducible E. coli expression vector, containing the arabinose inducible promoter P_{BAD} and regulator araC (Guzman (1995) J. Bact. 177:4121-4130), a kanamycin resistance gene Km(R) and the origin of replication ori327 from pBR322 (Watson (1988) Gene. 70:399-403). The E. coli host RV308 (laclq-, su-, ΔlacX74, gal IS II: OP308, strA, ebi.ac.uk/ena/data/view/ERP005879) with additional deletions in ampC and araB was transformed using chemical competent cells (Z-Competent cells, prepared with the Mix and Go!E. coli transformation kit, Zymo Research, Irvine CA, USA). Several clones from SEQ ID NO:1-6 were sequence verified and cultured in 2×PY containing 100 μg/ml neomycin (O/N). The preculture (1/100 vol) was used to inoculate the fermentation in MagicMedia™ E. coli expression medium (Thermo Fisher Scientific Inc), and 100 μg/ml neomycin (24 wells MTP, 3 ml volume, breathable seal, 550 RPM 80% RH), after 4 hours growth at 30° C., the cultures were induced with 0.02% arabinose (final concentration) and incubation was continued at 30° C. for 48 hours. Cell-pellets were isolated by high-speed centrifugation and frozen until further use. Cell free extract (CFE) was prepared by resuspending the frozen cell pellets and incubating for 1 hour at 37° C. with 1.2 ml lysis buffer (Tris-HCl 50 mM, DNasel 0.1 mg/ml, lysozyme 2 mg/ml, MgSO₄ 25 μM). Cell debris was removed by centrifugation and the CFE was stored at −20° C. until further characterization.

### Glucan Sucrases

The glucan sucrases used in this study were obtained from commercial suppliers (Sigma Aldrich for Leuconostoc mesenteroides glucan sucrase; NZYTech for Streptococcus mutans glucan sucrases)

All enzymes used in this study are mentioned in Table 1

TABLE 1
Enzymes used in this study.
Sucrose isomerases
Sequences appear after this Table

| | | | | Stock conc. |
|---|---|---|---|---|
| Uniprot ID | SEQ ID No | Donor organism | Name | (mg/ml) |
| D0VX20 | 1 | Protaminobacter rubrum* | Sis10 | 2.2 |
| Q6XNK6 | 2 | Pantoea dispersa | Sis2 | 1.0 |
| Q6XKX6 | 3 | Raoultella planticola | Sis12 | 1.7 |
| Q2PS28 | 4 | Pseudomonas mesoacidophila* | Sis4 | 2.0 |
| B5ABD8 | 5 | Enterobacter | Sis14 | 0.9 |
| S5YEW8 | 6 | Pectobacterium carotovorum | Sis15 | 0.7 |

Glucan sucrases

| | | | | Stock conc. |
|---|---|---|---|---|
| Identifier | Supplier | Donor organism | Name | (mg/ml) |
| D9909 | Sigma | Leuconostoc mesenteroides | B-1299 | 1.0 |
| SmGtf70B | NZYTech | Streptococcus mutans | 70B | 1.0 |
| SmGtf70C | NZYTech | Streptococcus mutans | 70C | 1.0 |
| SmGtf70D | NZYTech | Streptococcus mutans | 70D | 1.0 |

*P. rubrum most likely has to be renamed as Serratia plymuthica, and P. mesoacidophila was assigned as a Rhizobium species (Goulter et al. (2012) Enzyme Microb. Technol. 50, 57-64).

SEQUENCE ID NOS 1-6:

SEQ ID NO: 1
MTIPKWWKEAVFYQVYPRSFKDTNGDGIGDINGIIEKLDYLKALGIDAIWINPHYDSPNTD
NGYDIRDYRKIMKEYGTMEDFDRLISEMKKRNMRLMIDVVINHTSDQNEWFVKSKSSKD
NPYRGYYFWKDAKEGQAPNNYPSFFGGSAWQKDEKTNQYYLHYFAKQQPDLNWDNP
KVRQDLYAMLRFWLDKGVSGLRFDTVATYSKIPDFPNLTQQQLKNFAAEYTKGPNIHRY
VNEMNKEVLSHYDIATAGEIFGVPLDQSIKFFDRRRDELNIAFTFDLIRLDRDSDQRWRR
KDWKLSQFRQIIDNVDRTAGEYGWNAFFLDNHDNPRAVSHFGDDRPQWREPSAKALA
TLTLTQRATPFIYQGSELGMTNYPFKAIDEFDDIEVKGFWHDYVETGKVKADEFLQNVR
LTSRDNSRTPFQWDGSKNAGFTSGKPWFKVNPNYQEINAVSQVTQPDSVFNYYRQLIK
IRHDIPALTYGTYTDLDPANDSVYAYTRSLGAEKYLVVVNFKEQMMRYKLPDNLSIEKVII
DSNSKNVVKKNDSLLELKPWQSGVYKLNQ
SEQ ID NO: 2
MASPLTKPSTPIAATNIQKSADFPIWWKQAVFYQIYPRSFKDSNGDGIGDIPGIIEKLDYL
KMLGVDAIWINPHYESPNTDNGYDISDYRKIMKEYGSMADFDRLVAEMNKRGMRLMIDI
VINHTSDRHRWFVQSRSGKDNPYRDYYFWRDGKQGQAPNNYPSFFGGSAWQLDKQT
DQYYLHYFAPQQPDLNWDNPKVRAELYDILRFWLDKGVSGLRFDTVATFSKIPGFPDLS
KAQLKNFAEAYTEGPNIHKYIHEMNRQVLSKYNVATAGEIFGVPVSAMPDYFDRRREEL
NIAFTFDLIRLDRYPDQRWRRKPWTLSQFRQVISQTDRAAGEFGWNAFFLDNHDNPRQ
VSHFGDDSPQWRERSAKALATLLLTQRATPFIFQGAELGMTNYPFKNIEEFDDIEVKGF
WNDYVASGKVNAAEFLQEVRMTSRDNSRTPMQWNDSVNAGFTQGKPWFHLNPNYK
QINAAREVNKPDSVFSYYRQLINLRHQIPALTSGEYRDLDPQNNQVYAYTRILDNEKYLV
VVNFKPEQLHYALPDNLTIASSLLENVHQPSLQENASTLTLAPWQAGIYKLN
SEQ ID NO: 3
MAPSVNQNIHVHKESEYPAWWKEAVFYQIYPRSFKDTNDDGIGDIRGIIEKLDYLKSLGI
DAIWINPHYDSPNTDNGYDISNYRQIMKEYGTMEDFDNLVAEMKKRNMRLMIDVVINHT
SDQHPWFIQSKSDKNNPYRDYYFWRDGKDNQPPNNYPSFFGGSAWQKDAKSGQYYL
HYFARQQPDLNWDNPKVREDLYAMLRFWLDKGVSSMRFDTVATYSKIPGFPNLTPEQ
QKNFAEQYTMGPNIHRYIQEMNRKVLSRYDVATAGEIFGVPLDRSSQFFDPRRHELNM
AFMFDLIRLDRDSNERWRHKSWSLSQFRQIISKMDVTVGKYGWNTFFLDNHDNPRAVS
HFGDDRPQWREASAKALATITLTQRATPFIYQGSELGMTNYPFRQLNEFDDIEVKGFW
QDYVQSGKVTATEFLDNVRLTSRDNSRTPFQWNDTLNAGFTRGKPWFHINPNYVEINA
EREETREDSVLNYYKKMIQLRHHIPALVYGAYQDLNPQDNTVYAYTRTLGNERYLVVVN
FKEYPVRYTLPANDAIEEVVIDTQQQATAPHSTSLSLSPWQAGVYKLR
SEQ ID NO: 4
MEEAVKPGAPWWKSAVFYQVYPRSFKDTNGDGIGDFKGLTEKLDYLKGLGIDAIWINP
HYASPNTDNGYDISDYREVMKEYGTMEDFDRLMAELKKRGMRLMVDVVINHSSDQHE
WFKSSRASKDNPYRDYYFWRDGKDGHEPNNYPSFFGGSAWEKDPVTGQYYLHYFGR
QQPDLNWDTPKLREELYAMLRFWLDKGVSGMRFDTVATYSKTPGFPDLTPEQMKNFA
EAYTQGPNLHRYLQEMHEKVFDHYDAVTAGEIFGAPLNQVPLFIDSRRKELDMAFTFDL
IRYDRALDRWHTIPRTLADFRQTIDKVDAIAGEYGWNTFFLGNHDNPRAVSHFGDDRP
QWREASAKALATVTLTQRGTPFIFQGDELGMTNYPFKTLQDFDDIEVKGFFQDYVETG
KATAEELLTNVALTSRDNARTPFQWDDSANAGFTTGKPWLKVNPNYTEINAAREIGDPK
SVYSFYRNLISIRHETPALSTGSYRDIDPSNADVYAYTRSQDGETYLVVVNFKAEPRSFT
LPDGMHIAETLIESSSPAAPAAGAASLELQPWQSGIYKVK
SEQ ID NO: 5
MAYSAETSVTQSIQTQKESTLPAWWKEAVFYQIYPRSFKDTNGDGIGDIRGIIEKLDYLK
SLGIDAIWINPHYDSPNTDNGYDIRDYEKIMQEYGTMEDFDTLVSEMKKRNMRLMIDVVI
NHTSDQHPWFIQSKSSKENPYREYYFWRDGKDNQPPNNYPSFFGGSAWQKDDKTGQ
YYLHYFARQQPDLNWDNPKVRGDLYAMLRFWLDKGVSGMRFDTVATYSKIPGFPDLT
PEQQKNFAEQYTTGPNIHRYLQEMKQEVLSRYDVVTAGEIFGVPLERSSDFFDRRRNE
LDMSFMFDLIRLDRDSNERWRHKKWTLSQFRQIINKMDSNAGEYGWNTFFLDNHDNP
RAVSHFGDDSPQWIEPSAKALATIILTQRATPFIFQGSELGMTNYPFKKLNEFDDIEVKG
FWQDYVQTGKVSAEEFIDNVRLTSRDNSRTPFQWNDRKNAGFTSGKPWFRINPNYVEI
NADKELIRNDSVLNYYKEMIKLRHKTPALIYGTYKDISPEDDSVYAYTRTLGKERYLVVIN
FTEKTVRYPLPENNVIKSILIEANQNKTAEKQSTVLTLSPWQAGVYELQ
SEQ ID NO: 6
MATNHNEQDTKTVIAVNDGVSAHPVWWKEAVFYQVYPRSFKDSNGDGIGDLKGLTEK
LDYLKTLGINAIWINPHYDSPNTDNGYDIRDYRKIMKEYGTMDDFDNLIAEMKKRDMRL
MIDVVVNHTSNEHKWFVESKKSKDNPYRDYYIWRDGKDGTPPNNYPSFFGGSAWQKD
NVTQQYYLHYFGVQQPDLNWDNPKVREEVYDMLRFWIDKGVSGLRMDTVATFSKNPA
FPDLTPEQLKNFAYTYTQGPNLHRYIQEMHQKVLAKYDVVSAGEIFGVPLEEAAPFIDQ
RRKELDMAFSFDLIRLDRAVEERWRRNDWTLSQFRQINNRLVDMAGQYGWNTFFLSN
HDNPRAVSHFGDDRPEWRIRSAKALATLALTQRATPFIYQGDELGMTNYPFTSLSEFDD
IEVKGFWQDFVETGKVKPDVFLENVKQTSRDNSRTPFQWSNAEQAGFTTGTPWFRIN
PNYKNINAEDQTQNPDSIFHFYRQLIALRHATPAFTYGAYQDLDPNNNEVLAYTRELNQ
QRYLVVVNFKEKPVHYALPKTLSIKQTLLESGQKDKVAPNATSLELQPWQSGIYQLN

### Enzyme Quantification

SDS-PAGE followed by Coomassie staining was used to visualize the enzymes present in the samples. For the quantification of the individual enzymes, the focus was on bands of the correct molecular mass. Stain intensity of the bands was quantified by ImageQuant software. Protein stain intensity of the selected bands was compared to known quantities of bovine serum albumin (BSA) run on the same gel, and calculated back to the original enzyme stock solution

### Sugar Identification and Quantification

Sugar profile after incubation of sucrose containing solutions with the enzymes was analysed using the Dionex HPLC (HPAEC BioLC system, Dionex 5000) equipped with a CarboPac PA20 column. After incubating, the samples are diluted, filtered and the sugar components separated using the program described in Table 2.

TABLE 2
HPLC method used to separate sugars on the Dionex.

| | Time | NaOH | |
|---|---|---|---|
| Step | (min) | (mM) | Comment |
| 1 | −15 | 25 | Equilibration |
| 2 | 0 | 25 | Injection |
| 3 | 15 | 25 | Isocratic run |
| 4 | 30 | 30 | Slight gradient |
| 5 | 40 | 126 | Column wash (also with acetic acid) |
| 6 | 50 | 25 | Prepare for next run |

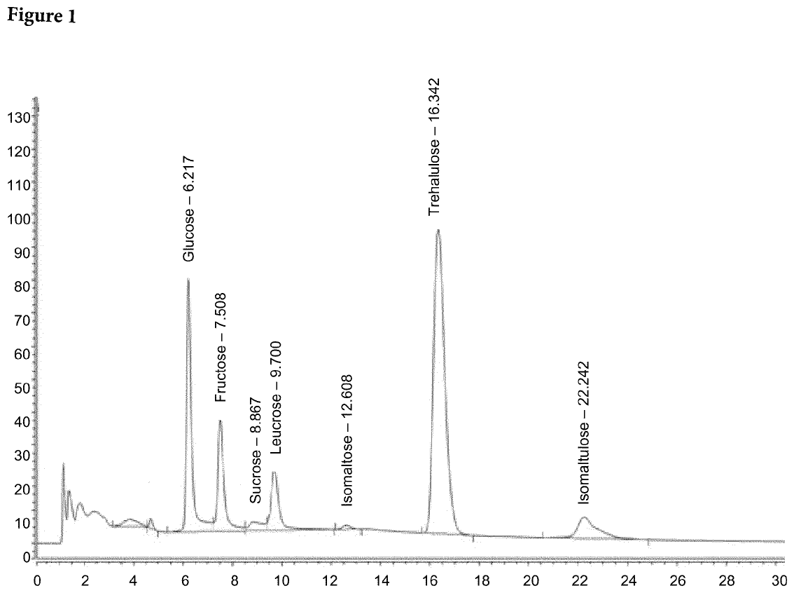

Peaks on HPLC were assigned and quantified by spiking pure solutions of sucrose, glucose, fructose, isomaltulose, leucrose, trehalulose and isomaltose (range of 2 to 75 mg/ml) obtained from Merck Millipore. An example of the separation of the different sugars using this technique is shown in FIG. 1. The response factor for each sugar was calculated from the integrated peak areas detected for the sugar concentrations and plotting a linear curve fit of the concentration versus the peak area. The response factor was used for the calculation of the absolute amount of the sugar present in each sample. Relative sugar concentration in percentage was calculated by dividing the absolute amount of each sugar measured in the sample, by the total amount of all sugars detected in the sample, and multiplied by 100.

### Calculation of Glycemic Index

Glycemic index (GI) in these experiments was calculated assuming a GI of the different sugars; Sucrose: 65; Fructose: 15; Glucose: 100; Isomaltulose: 32; Trehalulose: 32. Wolever (European Journal of Clinical Nutrition (2013) 67, 1229-1233) has stated that a GI>70 is regarded as high, and a Gl<55 is low, according to Canadian regulation. The percental content of each sugar in the product was divided by 100 and multiplied by its GI. All numbers were added up to calculate the GI of the different treated products.

### Example 2

### Activity of Sucrose Isomerases at Different pH

To test the activity of the different sucrose isomerases on sucrose at different pH, we incubated a 20% sucrose/250 mM sodium phosphate buffer with the different enzymes at 10% dilution (0.07-0.21 mg protein/ml). pH of the solution was set at either 4.5 and 6.0, and the incubation was for 6 hours at 37° C., after which the reaction was stopped by heating at 99° C. for 5 minutes. Conversion of sucrose into different sugars was quantified using the Dionex HPLC method. Results are depicted below in Table 3 as average percentage of the total amount of all sugars detected in the samples after the incubation, obtained from experiments with 2-4 different preparations of the respective enzymes.

TABLE 3
Conversion of sucrose into various sugars at various pHs using sucrose
isomerases

| Avg % | Sucrose | Fructose | Glucose | Isomaltulose | Isomaltose | Leucrose | Trehalulose |
|---|---|---|---|---|---|---|---|
| pH4.5 | | | | | | | |
| Sis10 | 1 | 18 | 17 | 37 | 5 | 1 | 21 |
| Sis2 | 0 | 16 | 20 | 49 | 3 | 1 | 12 |
| Sis12 | 4 | 2 | 4 | 64 | 1 | 1 | 26 |
| Sis4 | 0 | 9 | 14 | 22 | 0 | 9 | 46 |
| Sis14 | 1 | 0 | 3 | 63 | 0 | 0 | 33 |
| Sis15 | 0 | 1 | 4 | 33 | 0 | 1 | 61 |
| pH6.0 | | | | | | | |
| Sis10 | 4 | 28 | 23 | 10 | 9 | 3 | 24 |
| Sis2 | 3 | 10 | 13 | 63 | 1 | 3 | 7 |
| Sis12 | 4 | 3 | 4 | 60 | 0 | 1 | 28 |
| Sis4 | 2 | 8 | 11 | 20 | 0 | 11 | 47 |
| Sis14 | 1 | 0 | 4 | 60 | 0 | 0 | 35 |
| Sis15 | 1 | −1 | 2 | 30 | 0 | 2 | 66 |

From Table 3 it becomes clear that all tested enzymes are able to convert sucrose almost completely, at both pH6.0 and pH4.5. Product formation is somewhat dependent on the enzyme, but with all sucrose isomerase enzymes the most prominent products are isomaltulose and trehalulose, amounting to 60-100% of total sugars under most conditions.

### Example 3

### Activity of Glucan Sucrases at Different pH

To test the activity of the different glucan sucrases on sucrose at different pH, we incubated a 20% sucrose/250 mM sodium phosphate buffer with the different enzymes at 10% dilution. pH of the solution was set at either 4.5 and 6.0, and the incubation was for 6 hours at 37° C., after which the reaction was stopped by heating at 99° C. for 5 minutes. Sugar composition was quantified using the Dionex HPLC method. Results are depicted below in Table 4 as the percentage of total sugar after the conversion, obtained from experiments with the respective enzymes. Since glucan can exist of different forms, it is difficult to quantify using the HPLC method used in these experiments. Therefore, the total formation of glucan was calculated from the difference in the increase in fructose and glucose, after correction for the fructose and glucose content of the blanc without added enzyme. Therefore, the numbers indicated are only a rough estimate of the total amount of glucan formed.

TABLE 4
Conversion of sucrose into various sugars at various pHs using glucan
sucrases

| Avg.% | Sucrose | Fructose | Glucose | Isomaltulose | Isomaltose | Leucrose | Trehalulose | Glucan |
|---|---|---|---|---|---|---|---|---|
| pH4.5 | | | | | | | | |
| B-1299 | 90 | 1 | −1 | 4 | 0 | 0 | 0 | 7 |
| 70B | 9 | 43 | 6 | −1 | 0 | 0 | 2 | 41 |
| 700 | 3 | 40 | 5 | 0 | 0 | 11 | 2 | 39 |
| 70D | 100 | −5 | −1 | 5 | 0 | 0 | 0 | 1 |
| blanc | 106 | −5 | −1 | 0 | 0 | 0 | 0 | 0 |
| pH6.0 | | | | | | | | |
| B-1299 | 97 | 0 | −1 | 0 | 0 | 0 | 0 | 4 |
| 70B | 5 | 34 | 4 | 7 | 0 | 14 | 2 | 34 |
| 70C | 1 | 41 | 5 | 0 | 0 | 13 | 1 | 40 |
| 70D | 106 | −5 | −1 | 0 | 0 | 0 | 0 | 1 |
| blanc | 104 | −5 | −1 | 3 | 0 | 0 | 0 | 0 |

From Table 4 it becomes clear that some of the glucan sucrases (70B and 70C) are able to convert almost all sucrose at both pH6.0 and pH4.5, while the others do show very little activity. Product formation is somewhat dependent on the enzyme, and glucan yield is maximally approximately 30-40% under these conditions. Other sugars that are formed by these enzymes are mainly leucrose and some isomaltulose.

### Example 4

### Activity of Sucrose Isomerases in Cola

The activity of the sucrose isomerases and glucan sucrases was tested in cola (Coca-Cola®; local supermarket). For this experiment the enzymes were again added at 10% dilution in this matrix, and incubated for 130 minutes at 37° C., after which the reaction was stopped by heating at 90° C. for 5 minutes. Approximately 100 g/L total sugar is measured in the cola. Since cola is very acidic (pH 2.6) part of the sucrose is inverted into glucose and fructose, especially during the heating step, and the total amount of sucrose measured is probably lower, and the amount of fructose and glucose higher, then present in cola. Again, conversion of sucrose into different sugars was quantified using the Dionex HPLC method. Results are depicted below as average percentage of the total amount of all sugars detected in the samples after the incubation. As shown in Table 5 below, 40-50% of total sugar can be converted into the low-glycemic sugars isomaltulose and trehalulose using Sis14 and Sis15 sucrose isomerases, leading to a low glycemic index. Sis14 seems to have a preference for the formation of isomaltulose, while Sis15 has a preference for trehalulose formation, as was already seen in the buffer experiment of Example 2. Sis2 did not show any activity in cola, while Sis10, Sis12 and Sis4 showed 8-15% conversion.

TABLE 5
sugar conversion in cola

| | Su- | Fruc- | Glu- | Iso- | Trehalu- | Glycemic |
|---|---|---|---|---|---|---|
| % cola | crose | tose | cose | maltulose | lose | index |
| Sis10 | 42 | 23 | 28 | 7 | 1 | 61 |
| Sis2 | 49 | 23 | 28 | 0 | 0 | 63 |
| Sis12 | 39 | 22 | 27 | 9 | 4 | 60 |
| Sis4 | 35 | 22 | 28 | 5 | 10 | 59 |
| Sis14 | 2 | 26 | 31 | 28 | 13 | 49 |
| Sis15 | 2 | 24 | 28 | 18 | 28 | 48 |
| Control | 50 | 23 | 27 | 0 | 0 | 63 |

None of the glucan sucrases showed significant activity in cola

### Example 5

### Activity of Sucrose Isomerases in Chocolate Milk at Simulated Stomach Conditions

The activity of the sucrose isomerases and glucan sucrases was tested in chocolate milk. Skimmed chocolate milk (Friesland Campina) has a neutral pH (pH6.4) and contains approximately 100 g/L sucrose. In this experiment 100 ml chocolate milk was incubated under agitation at 20 rpm in a water bath set at 37° C., and the pH was decreased in steps by the addition of HCl. Pepsin from porcine gastric mucosa powder >250 u/mg solid (Sigma; P7000) was added at 0.02 mg/ml final concentration, and sucrose isomerases were added at 0.1% (v/v), at the start of the experiment. After incubation for 0.5 hour, the pH was set at 4.0, after 1 hour set to pH3.0 and after 1.5 hours to pH2.0 by addition of HCl. 1 ml samples were withdrawn after 5-10 minutes (t=0), 1 hour (t=1) and 2 hours incubation (t=2) and the enzymatic activity was immediately inactivated by heating (99° C. for 5 minutes).

Samples were analyzed using the HPLC method as described above, and the different sugars were quantified and expressed as percentage of the total amount of all sugars detected in the samples, and are shown below in Table 6. Again, as also observed in the cola experiment, the samples showed (chemical) conversion of sucrose into glucose and fructose by heating at low pH (especially prevalent in the t=2 samples).

TABLE 6
Sugar conversion in chocolate milk

| % | | Su- | Fruc- | Glu- | Isomalt- | Trehalu- | Glycemic |
|---|---|---|---|---|---|---|---|
| choco | time | crose | tose | cose | ulose | lose | index |
| Sis10 | t = 0 | 77 | 9 | −2 | 17 | 0 | 54 |
| | t = 1 | 54 | 12 | 0 | 33 | 1 | 48 |
| | t = 2 | 14 | 24 | 20 | 40 | 1 | 46 |
| Sis2 | t = 0 | 96 | 11 | −3 | −3 | 0 | 59 |
| | t = 1 | n.d. | n.d. | n.d. | n.d. | n.d. | n.d. |
| | t = 2 | 62 | 23 | 14 | 1 | 0 | 58 |
| Sis12 | t = 0 | 97 | 11 | −3 | −7 | 1 | 60 |
| | t = 1 | 65 | 13 | 0 | 10 | 13 | 51 |
| | t = 2 | 24 | 29 | 23 | 11 | 13 | 51 |
| Sis4 | t = 0 | 82 | 10 | −3 | 5 | 6 | 55 |
| | t = 1 | 24 | 10 | −2 | 22 | 46 | 37 |
| | t = 2 | 4 | 15 | 6 | 33 | 42 | 35 |
| Sis14 | t = 0 | 101 | 13 | −4 | −10 | 0 | 60 |
| | t = 1 | 82 | 15 | 2 | −1 | 2 | 58 |
| | t = 2 | 15 | 41 | 41 | 0 | 2 | 58 |
| Sis15 | t = 0 | 103 | 6 | −4 | −7 | 2 | 63 |
| | t = 1 | 58 | 12 | −2 | 7 | 24 | 48 |
| | t = 2 | 20 | 28 | 23 | 8 | 21 | 50 |
| Con- | t = 0 | 103 | 0 | −3 | 0 | 0 | 64 |
| trol | t = 1 | 86 | 13 | 1 | 0 | 0 | 59 |
| | t = 2 | 43 | 31 | 27 | 0 | 0 | 59 |

From Table 6 it becomes clear that especially Sis4 is able to convert up to 75% of total sucrose in chocolate milk into low-glycemic sugars like isomaltulose and trehalulose, under simulated stomach conditions. Sis10 can convert ˜40% of the sucrose into isomaltulose specifically, while Sis15 produces ˜30% total of mainly trehalulose and Sis12 ˜20% total of both sucrose isomers. Sis2 and Sis14 did not show an effect in this experiment. So even with a much lower enzyme dosage at conditions mimicking stomach digestion, most of these enzymes can lead to a lowering of the glycemic index of a regular food product like chocolate milk.

None of the glucan sucrases showed significant activity in chocolate milk in this experiment.

### Example 6

### Activity of Sucrose Isomerases in Ice Cream at Simulated Stomach Conditions

The activity of the sucrose isomerases and glucan sucrases was tested in ice cream. Ice cream (Albert Heijn Roomijs vanilla) has a neutral pH (pH6.5) and contains approximately 230 g/kg sucrose. The experiment was performed exactly as was described in Example 4 including the enzyme dosage, pepsin addition, pH setting, sampling times and amounts and sugar analysis on HPLC.

Again, the different sugars were quantified and expressed as percentage of the total amount of sugar detected in the samples. Results of the sugar analysis are depicted in Table 7 below.

TABLE 7
Conversion of sugars in ice cream

| % ice | | Su- | Fruc- | Glu- | Iso- | Trehalu- | Glycemic |
|---|---|---|---|---|---|---|---|
| cream | time | crose | tose | cose | maltulose | lose | index |
| Sis10 | t = 0 | 97 | 4 | −1 | 0 | 0 | 63 |
| | t = 1 | 73 | 7 | 2 | 16 | 2 | 56 |
| | t = 2 | 32 | 26 | 28 | 13 | 1 | 57 |
| Sis2 | t = 0 | 98 | 5 | 0 | −3 | 0 | 64 |
| | t = 1 | 96 | 5 | −1 | 0 | 0 | 62 |
| | t = 2 | 38 | 29 | 30 | 3 | 0 | 60 |
| Sis12 | t = 0 | 101 | 0 | −1 | 0 | 0 | 65 |
| | t = 1 | 83 | 5 | 0 | 8 | 5 | 58 |
| | t = 2 | 19 | 33 | 38 | 6 | 4 | 59 |
| Sis4 | t = 0 | 62 | 4 | −1 | 7 | 28 | 51 |
| | t = 1 | 63 | 8 | 4 | 2 | 22 | 54 |
| | t = 2 | 13 | 29 | 31 | 5 | 22 | 52 |
| Sis14 | t = 0 | 95 | 5 | 0 | 0 | 0 | 63 |
| | t = 1 | 69 | 16 | 15 | −1 | 1 | 62 |
| | t = 2 | 44 | 27 | 29 | 0 | 1 | 62 |
| Sis15 | t = 0 | 96 | 5 | 9 | 0 | 0 | 62 |
| | t = 1 | 82 | 6 | 0 | 4 | 8 | 58 |
| | t = 2 | 37 | 25 | 29 | 3 | 6 | 60 |
| Control | t = 0 | 95 | 5 | 0 | 0 | 0 | 62 |
| | t = 1 | 93 | 6 | 1 | 0 | 0 | 62 |
| | t = 2 | 16 | 39 | 45 | 0 | 0 | 61 |

From Table 7 it becomes clear that Sis4 is able to convert up to 25-35% of total sucrose in ice cream into low-glycemic sugars like isomaltulose and trehalulose, under simulated stomach conditions. Sis10 can convert ˜15% of the sucrose into isomaltulose specifically, and also Sis 12 and Sis15 produce some sucrose isomers. Again, Sis2 and Sis14 had no activity under these conditions. So even with a much lower enzyme dosage at conditions mimicking stomach digestion, most of these enzymes can lead to a lowering of the glycemic index of a regular food product like ice cream.

None of the glucan sucrases showed significant activity in ice cream in this experiment.

1. A method of lowering the increase of blood glucose levels in an animal, including a human ingesting sucrose, comprising administering a nutraceutical, pharmaceutical or nutritional supplement comprising a sucrose isomerase to the animal including a human in need thereof.

2. The method according to claim 1, wherein the nutraceutical, pharmaceutical or nutritional supplement is suitable for administration to humans.

3. The method according to claim 1, wherein the nutraceutical, pharmaceutical or nutritional supplement is suitable for administration to animals.

4. The method according to claim 1, wherein the sucrose isomerase comprises an amino acid sequence having at least 90% identity to any one of the sequences of SEQ ID NO: 1-6.

5. The method according to claim 4, wherein the sucrose isomerase comprises an amino acid sequence having at least 90% identity to any one of the sequences of SEQ ID NO: 4.

6. A method of lowering the glycemic index of a food or feed which is consumed by a human or companion animal comprising administering to the human or companion animal a nutraceutical, pharmaceutical or nutritional supplement comprising a sucrose isomerase.

7. The method according to claim 6, wherein the nutraceutical, pharmaceutical or nutritional supplement is suitable for administration to humans.

8. The method according to claim 6, wherein the nutraceutical, pharmaceutical or nutritional supplement is suitable for companion administration to animals.

9. The method according to claim 6, wherein the sucrose isomerase comprises an amino acid sequence having at least 90% identity to any one of the sequences of SEQ ID NO: 1-6.

10. The method according to claim 9, wherein the sucrose isomerase comprises an amino acid sequence having at least 90% identity to any one of the sequences of SEQ ID NO: 4.

11. A method of losing weight or maintaining weight loss in a human or companion animal comprising administering to the human or companion animal a nutraceutical, pharmaceutical or nutritional supplement comprising a sucrose isomerase.

12. The method according to claim 11, wherein the nutraceutical, pharmaceutical or nutritional supplement is suitable for administration to humans.

13. The method according to claim 11, wherein the nutraceutical, pharmaceutical or nutritional supplement is suitable for administration to companion animals.

14. The method according to claim 11, wherein the sucrose isomerase comprises an amino acid sequence having at least 90% identity to any one of the sequences of SEQ ID NO: 1-6.

15. The method according to claim 14, wherein the sucrose isomerase comprises an amino acid sequence having at least 90% identity to any one of the sequences of SEQ ID NO: 4.
